(19) United States
(12) Patent Application Publication
(10) Pub. No.: US 2025/0128076 A1
(43) Pub. Date: Apr. 24, 2025

(54) Weighted Stimulation Field Models for Programming Deep Brain Stimulation

(71) Applicant: Boston Scientific Neuromodulation Corporation, Valencia, CA (US)

(72) Inventor: León M. Juárez Paz, Berlin (DE)

(21) Appl. No.: 18/915,065

(22) Filed: Oct. 14, 2024

Related U.S. Application Data
(60) Provisional application No. 63/592,820, filed on Oct. 24, 2023.

(51) Int. Cl. A61N 1/372; A61N 1/05

(52) U.S. Cl. CPC A61N 1/37241; A61N 1/37247; A61N 1/0534

(57) ABSTRACT

Methods and systems for assisting the programming of stimulation parameters for deep brain stimulation (DBS) of a subject patient are described. An accumulated database catalogues stimulation parameter sets used in programming sessions for a plurality of patients and correlates the parameter sets with good therapeutic results and/or with side effects. The accumulated database also includes stimulation field models (SFMs) for the parameter sets. Overlap of the SFMs for parameter sets may be correlated with therapeutic effects and used to determine target volumes and or avoidance volumes for the stimulation of the subject patient. In some embodiments, the SFMs may be weighted to de-emphasize volumes proximate to the electrode lead.

### CROSS REFERENCE TO RELATED APPLICATIONS

This is a non-provisional application of U.S. Provisional Patent Application Ser. No. 63/592,820, filed Oct. 24, 2023, which is incorporated herein by reference in its entirety, and to which priority is claimed.

### FIELD OF THE INVENTION

This application relates to deep brain stimulation (DBS), and more particularly, to methods and systems for optimizing DBS stimulation parameters.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease, essential tremor, dystonia, and epilepsy, to name but a few. Further details discussing the treatment of diseases using DBS are disclosed in U.S. Pat. Nos. 6,845,267, and 6,950,707.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus (STN) has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the STN. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the STN may occur due to non-selective activation of non-motor pathways within or around the STN.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads, but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system, or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads, or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s). There is a need for methods and systems that assist a clinician in determining optimum stimulation parameters for treating the patient.

### SUMMARY

Disclosed here are methods for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising: receiving accumulated data from a database, wherein the accumulated data comprises: previous trial stimulation parameter sets provided to previous patients or the subject patient, stimulation field models (SFMs) for each of the trial stimulation parameter sets, and scores indicative of the therapeutic effectiveness of each of the trial stimulation parameter sets for the previous patients, aggregating the SFMs of each of the trial stimulation parameter sets having a score exceeding a threshold score or within a defined threshold range, determining an overlap of the aggregated SFMs, and using the overlap to determine electrical stimulation parameters for the subject patient. According to some embodiments, aggregating the SFMs comprise voxelizing the SFMs having a score exceeding the threshold score and overlaying the voxelized SFMs in a common voxel space. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space. According to some embodiments, the method further comprises associating a weighting value with each of the voxels of the voxelized SFMs. According to some embodiments, associating a weighting value with each of the voxels of the voxelized SFMs comprises operating on the voxelized SFMs using a weighting function. According to some embodiments, the weighting function is a kernel weighting function. According to some embodiments, each of the voxelized SFMs comprise voxels associated peripheral regions of the SFM and voxels associated with regions near an electrode lead from which the SFM emanates. According to some embodiments, the weighting values associated with voxels associated peripheral regions of the SFM are greater than the weighting values associated with voxels associated with regions near an electrode lead from which the SFM emanates. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space and of the weighting values associated with corresponding voxels of the aggregated SFMs. According to some embodiments, the method further comprises displaying an indication of the overlap of the aggregated SFMs on a graphical user interface (GUI). According to some embodiments, using the overlap to determine electrical stimulation parameters for the subject patient comprises determining stimulation parameters that provide stimulation to a region where the aggregated SFMs overlap. According to some embodiments, using the overlap to determine electrical stimulation parameters for the subject patient comprises determining stimulation parameters that provide stimulation to a region where the overlap of the aggregated SFMs exceed a threshold.

Also disclosed herein is a system for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the system comprising: an external computing device comprising control circuitry configured to perform a method, the method comprising: receiving accumulated data from a database, wherein the accumulated data comprises: previous trial stimulation parameter sets provided to previous patients or the subject patient, stimulation field models (SFMs) for each of the trial stimulation parameter sets, and scores indicative of the therapeutic effectiveness of each of the trial stimulation parameter sets for the previous patients, aggregating the SFMs of each of the trial stimulation parameter sets having a score exceeding a threshold score or within a defined threshold range, determining an overlap of the aggregated SFMs, and using the overlap to determine electrical stimulation parameters for the subject patient. According to some embodiments, aggregating the SFMs comprise voxelizing the SFMs having a score exceeding the threshold score and overlaying the voxelized SFMs in a common voxel space. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space. According to some embodiments, the method further comprises associating a weighting value with each of the voxels of the voxelized SFMs. According to some embodiments, associating a weighting value with each of the voxels of the voxelized SFMs comprises operating on the voxelized SFMs using a weighting function. According to some embodiments, the weighting function is a kernel weighting function. According to some embodiments, each of the voxelized SFMs comprise voxels associated peripheral regions of the SFM and voxels associated with regions near an electrode lead from which the SFM emanates. According to some embodiments, the weighting values associated with voxels associated peripheral regions of the SFM are greater than the weighting values associated with voxels associated with regions near an electrode lead from which the SFM emanates. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space and of the weighting values associated with corresponding voxels of the aggregated SFMs. According to some embodiments, the method further comprises displaying an indication of the overlap of the aggregated SFMs on a graphical user interface (GUI). According to some embodiments, using the overlap to determine electrical stimulation parameters for the subject patient comprises determining stimulation parameters that provide stimulation to a region where the aggregated SFMs overlap. According to some embodiments, using the overlap to determine electrical stimulation parameters for the subject patient comprises determining stimulation parameters that provide stimulation to a region where the overlap of the aggregated SFMs exceed a threshold.

Also disclosed herein is a method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising: receiving accumulated data from a database, wherein the accumulated data comprises: trial stimulation parameter sets provided to previous patients that are not the subject patient, stimulation field models (SFMs) for each of the trial stimulation parameter sets, and scores indicative of side effects of each of the trial stimulation parameter sets for the previous patients, aggregating the SFMs of each of the trial stimulation parameter sets having a score exceeding a threshold score, determining an overlap of the aggregated SFMs, and using the overlap to determine electrical stimulation parameters for the subject patient.

Also disclosed herein is a non-volatile computer readable medium comprising instructions for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, wherein the non-volatile computer readable medium comprises instructions, which when executed on a computing device, configure the computing device to perform a method comprising: receiving accumulated data from a database, wherein the accumulated data comprises: previous trial stimulation parameter sets provided to previous patients or the subject patient, stimulation field models (SFMs) for each of the trial stimulation parameter sets, and scores indicative of the therapeutic effectiveness of each of the trial stimulation parameter sets for the previous patients, aggregating the SFMs of each of the trial stimulation parameter sets having a score exceeding a threshold score or within a defined threshold range, determining an overlap of the aggregated SFMs, and using the overlap to determine electrical stimulation parameters for the subject patient. According to some embodiments, the SFMs comprise voxelizing the SFMs having a score exceeding the threshold score and overlaying the voxelized SFMs in a common voxel space. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space. According to some embodiments, the method further comprises associating a weighting value with each of the voxels of the voxelized SFMs. According to some embodiments, associating a weighting value with each of the voxels of the voxelized SFMs comprises operating on the voxelized SFMs using a weighting function. According to some embodiments, the weighting function is a kernel weighting function. According to some embodiments, each of the voxelized SFMs comprise voxels associated peripheral regions of the SFM and voxels associated with regions near an electrode lead from which the SFM emanates. According to some embodiments, the weighting values associated with voxels associated peripheral regions of the SFM are greater than the weighting values associated with voxels associated with regions near an electrode lead from which the SFM emanates. According to some embodiments, determining an overlap of the aggregated SFMs comprises determining overlap values for each of the voxels of the common voxel space, wherein the overlap values for each of the voxels are indicative of the number of aggregated SFMs occupying that voxel of the common voxel space and of the weighting values associated with corresponding voxels of the aggregated SFMs. According to some embodiments, the method further comprises displaying an indication of the overlap of the aggregated SFMs on a graphical user interface (GUI). According to some embodiments, using the overlap to determine electrical stimulation parameters for the subject patient comprises determining stimulation parameters that provide stimulation to a region where the aggregated SFMs overlap. According to some embodiments, using the overlap to determine electrical stimulation parameters for the subject patient comprises determining stimulation parameters that provide stimulation to a region where the overlap of the aggregated SFMs exceed a threshold.

The invention may also reside in the form of a programed external device, such as a clinician programmer or other computing device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIG. 1A shows an Implantable Pulse Generator (IPG).

FIG. 1B shows a percutaneous lead having split-ring electrodes.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment useable to provide stimulation before implantation of an IPG.

FIG. 5 shows various external devices capable of communicating with and programming stimulation in an IPG or ETS.

FIG. 6 illustrates an embodiment of a user interface (UI) for programming stimulation.

FIG. 7 illustrates an embodiment of a system for optimizing stimulation for DBS.

FIG. 8 illustrates an accumulated database.

FIG. 9 illustrates a workflow for using overlap of SFMs from an accumulated database to optimize stimulation parameters for a subject patient.

FIGS. 10A and 10B illustrate the overlap of SFMs associated with therapeutic effects and SFMs associated with side effects, respectively.

FIGS. 11A and 11B show examples of a raw SFM and a weighted SFM, respectively.

FIGS. 12A and 12B show overlaps of weighted SFMs associated with therapeutic effects and SFMs associated with side effects, respectively.

### DETAILED DESCRIPTION

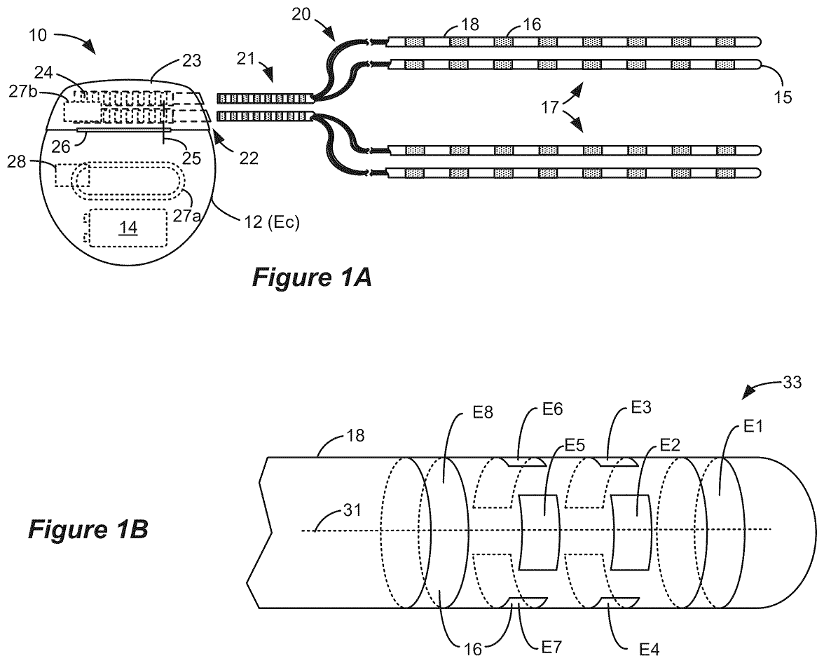

A DBS system typically includes an Implantable Pulse Generator (IPG) 10 shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more electrode leads 15 can be used having ring-shaped electrodes 16 carried on a flexible body 18.

In yet another example shown in FIG. 1B, an electrode lead 33 can include one or more split-ring electrodes. In this example, eight electrodes 16 (E1-E8) are shown. Electrode E1 at the distal end of the lead and electrode E8 at a proximal end of the lead comprise ring electrodes spanning 360 degrees around a central axis of the lead 33. In some embodiments, the electrode E1 may be a “bullet tip” electrode, meaning that it can cover the tip of the electrode lead. Electrodes E2, E3, and E4 comprise split-ring electrodes, each of which are located at the same longitudinal position along the central axis 31, but with each spanning less than 360 degrees around the axis. For example, each of electrodes E2, E3, and E4 may span 90 degrees around the axis 31, with each being separated from the others by gaps of 30 degrees. Electrodes E5, E6, and E7 also comprise split-ring electrodes, but are located at a different longitudinal position along the central axis 31 than are split ring electrodes E4, E2, and E3. As shown, the split-ring electrodes E2-E4 and E5-E7 may be located at longitudinal positions along the axis 31 between ring electrodes E1 and E8. However, this is just one example of a lead 33 having split-ring electrodes. In other designs, all electrodes can be split-ring, or there could be different numbers of split-ring electrodes at each longitudinal position (i.e., more or less than three), or the ring and split-ring electrodes could occur at different or random longitudinal positions, etc.

Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 15, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Lead wires 20 are tunneled through the neck and the scalp and the electrode leads 15 (or 33) are implanted through holes drilled in the skull and positioned for example in the subthalamic nucleus (STN). IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

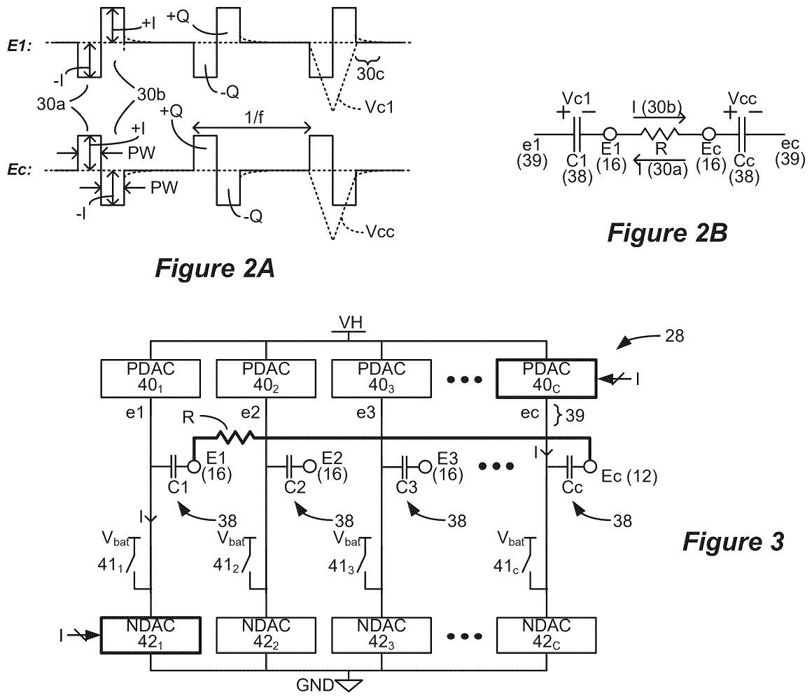

Stimulation in IPG 10 is typically provided by pulses each of which may include a number of phases such as 30a and 30b, as shown in the example of FIG. 2A. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude+I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrodes can be changed: Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40; and one or more current sinks 42ᵢ. The sources and sinks 40; and 42; can comprise Digital-to-Analog converters (DACs), and may be referred to as PDACs 40; and NDACs 42; in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node Ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40; and NDACs 42; allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40; and the electrode nodes ei 39, and between the one or more NDACs 42; and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes.

Much of the stimulation circuitry 28 of FIG. 3, including the PDACs 40; and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors C1 and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors C1 and Cc is actively recovered, and thus voltages Vc1 and Vcc increase and return to 0V at the end of the second pulse phase 30b.

To recover all charge by the end of the second pulse phase 30b of each pulse (Vc1=Vcc=0V), the first and second phases 30a and 30b are charged balanced at each electrode, with the first pulse phase 30a providing a charge of −Q(−*PW) and the second pulse phase 30b providing a charge of +Q(+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41i, which are described further in U.S. Patent Application Publications 2018/0071527 and 2018/0140831. Passive recovery switches 41; may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41i. As shown in FIG. 3, the other end of the switches 41; not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

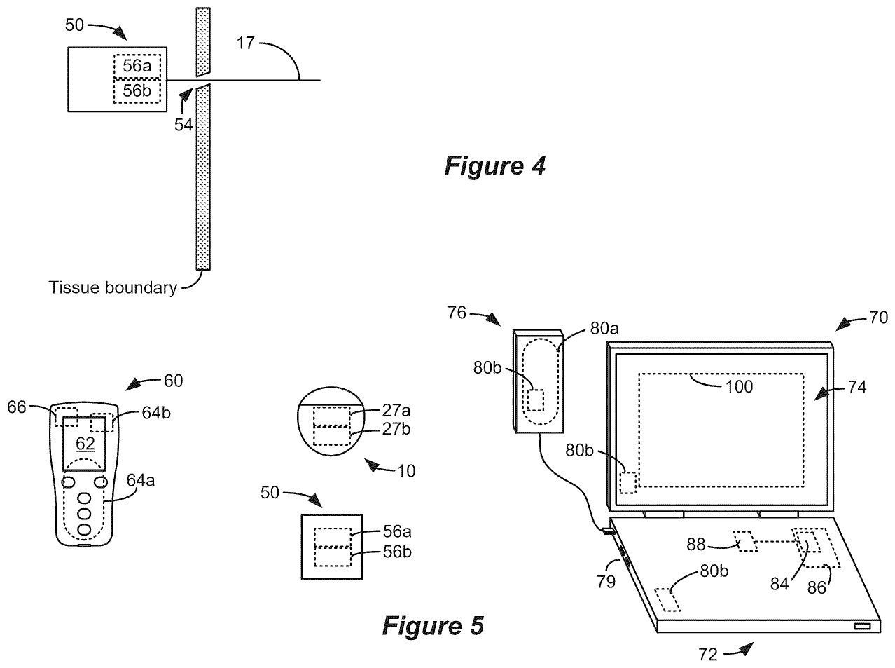

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 100 provided on the display 74 of the computing device 72. As one skilled in the art understands, the GUI 100 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 100, can also enable communications via antennas 80a or 80b to communicate stimulation parameters chosen through the GUI 100 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

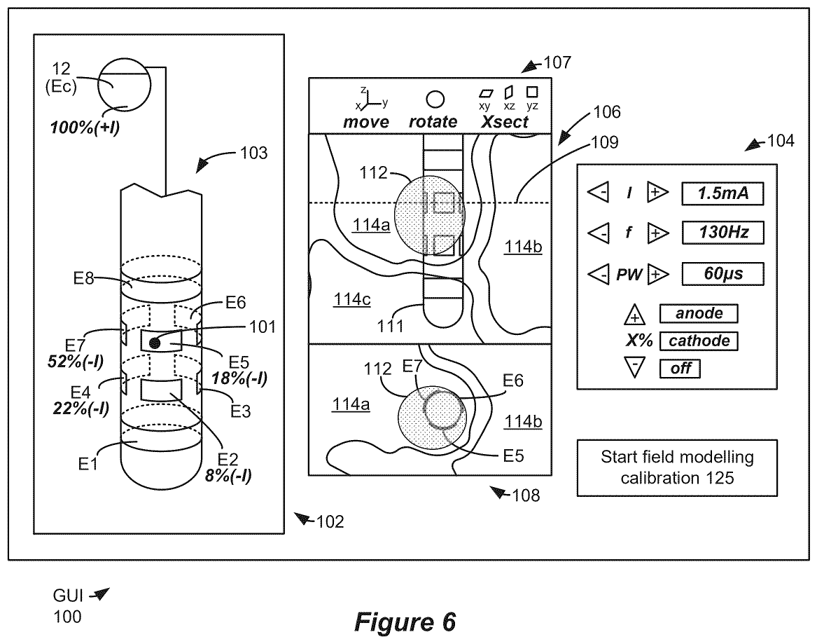

Particularly in the DBS context, it can be useful to provide a clinician with a visual indication of how stimulation selected for a patient will interact with the tissue in which the electrodes are implanted. This is illustrated in FIG. 6, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads, or after implantation when a therapeutically useful stimulation program is being chosen for a patient. However, GUI 100 could be rendered on a patient external programmer 60 (FIG. 5) or any other external device capable of communicating with the IPG 10 or ETS 50.

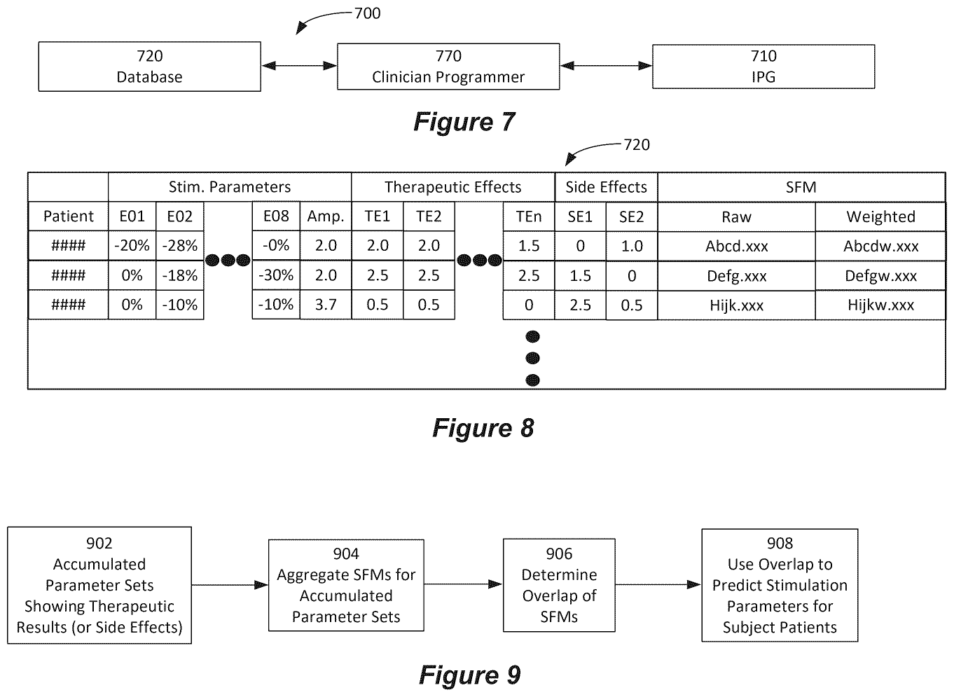

GUI 100 allows a clinician (or patient) to select the stimulation program that the IPG 110 or ETS 50 will provide and provides options that control sensing of innate or evoked responses, as described below. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. See, e.g., U.S. Patent Application Publication 2018/0071513. Nonetheless, interface 104 is simply shown for simplicity in FIG. 7 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 102, which can include an image 103 of the one or more leads that have been implanted in the patient. Although not shown, the leads interface 102 can include a selection to access a library of relevant images 103 of the types of leads that may be implanted in different patients.

In the example shown in FIG. 6, the leads interface 102 shows an image 103 of a single split-ring lead 33 like that described earlier with respect to FIG. 1B. The leads interface 102 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current+I or −I that each selected electrode will receive to be specified in terms of a percentage, X. For example, in FIG. 6, the case electrode 12 Ec is specified to receive X=100% of the current I as an anodic current+I. The corresponding cathodic current −I is split between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be shaped. The currents specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as a stimulation field model (SFM) 112 (also referred to herein as a volume of tissue activated (VTA)) formed using the selected stimulation parameters. The SFM 112 is formed by field modelling, for example, in the clinician programmer 70. An example of creating an SFM based on stimulation parameters is discussed in U.S. Pat. No. 8,849,411, “USER-DEFINED GRAPHICAL SHAPES USED AS A VISUALIZATION AID FOR STIMULATOR PROGRAMMING”, assigned to Boston Scientific Neuromodulation Corporation, which is herein incorporated by reference in its entirety. Other examples of such modeling and volume estimation are discussed in U.S. Pat. No. 8,190,250 B2, entitled “SYSTEM AND METHOD FOR ESTIMATING VOLUME OF ACTIVATION IN TISSUE”, U.S. Pat. No. 8,706,250 B2, entitled “NEUROSTIMULATION SYSTEM FOR IMPLEMENTING MODEL-BASED ESTIMATE OF NEUROSTIMULATION EFFECTS”, U.S. Pat. No. 8,934,979 B2, entitled “NEUROSTIMULATION SYSTEM FOR SELECTIVELY ESTIMATING VOLUME OF ACTIVATION AND PROVIDING THERAPY”, U.S. Pat. No. 9,792,412 B2, entitled “SYSTEMS AND METHODS FOR VOA MODEL GENERATION AND USE”, all assigned to Boston Scientific Neuromodulation Corporation, which are incorporated by reference herein in their entirety. The illustrated embodiment of the GUI 99 includes a selection option 125 for initiating such modeling. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 6 for the patient in question, which tissue structures may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. As one skilled in the art will understand, the location of the lead(s) can be precisely referenced to the tissue structures 114i because the lead(s) are implanted using a stereotactic frame (not shown). This allows the clinician programmer 70 on which GUI 100 is rendered to overlay the lead image 111 and the SFM 112 with the tissue imaging information in the visualization interface 106 so that the position of the SFM 112 relative to the various tissue structures 114i can be visualized. The image of the patient's tissue may also be taken after implantation of the lead(s), or tissue imaging information may comprise a generic image pulled from a library which is not specific to the patient in question, in some embodiments.

The various images shown in the visualization interface 106 (i.e., the lead image 111, the SFM 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the SFM 112 can allow the user to get a sense of the strength and reach of the stimulation at different locations. Although GUI 100 includes stimulation definition (102, 104) and imaging (108, 106) in a single screen of the GUI, these aspects can also be separated as part of the GUI 100 and made accessible through various menu selections, etc.

Especially in a DBS application, it is important that correct stimulation parameters be determined for a given patient. Improper stimulation parameters may not yield effective relief of a patient's symptoms, or may cause unwanted side effects. To determine proper stimulation, a clinician typically uses a GUI such as GUI 100 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores(S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient.

This disclosure relates to methods and systems for optimizing stimulation parameters for a DBS patient. In embodiments, quantitative objective information relating the position of the electrode lead with respect to anatomical features of the patient's brain are used to predict stimulation parameters that should be effective for the patient and/or that are likely to cause side effects for the patient. According to some embodiments, the electrode leads may be implanted in, or near, a target brain structure, such as the patient's STN. Other brain structure targets may include the Globus Pallidus Internus, the Ventral Intermediate Nucleus, or other targets, as known in the art. FIG. 7 illustrates a simplified overview 700 of a system for performing aspects of the disclosed methods. The illustrated system comprises a clinician programmer (CP) 770, which may include the features described above with respect to the CP 70 (FIG. 5). The CP 770 may comprise control circuitry configured to perform aspects of the disclosed methods. According to some embodiments, the CP 770 comprises non-transitory computer readable computer code, which when executed by the CP 770, configures the CP for performing aspects of the disclosed methods. The CP 770 may be configured to communicate an IPG 710, which may include the features described above with respect to the IPG 10 (FIGS. 1A and 5). The CP 770 may also be configured with access to a database 720, which is referred to herein as an “accumulated database.” The features of the database 720 will be apparent based on the below discussion. The database 720 may be comprised within the CP 770, for example, within computer-readable storage.

Alternatively (or additionally), aspects of the database 720 may be configured external to the CP 770, for example, on a local or remote external server. In such cases, the CP 770 may communicate with the database 720, for example, via an internet connection.

FIG. 8 illustrates an embodiment of an accumulated database 720. The accumulated database 720 comprises data collected from fitting sessions conducted for a plurality of patients (and, possibly, for multiple hemispheres of the patients). The fitting data from the various fitting sessions comprises data relating to the stimulation parameters that were applied to the patient. The stimulation parameters may comprise amplitude, stimulation field energy, frequency, pulse width, duty cycle, electrode configuration, or the like, may be collated in the database. In the illustrated example, amplitude (Amp.) and electrode configuration (i.e., the percentage of current delivered at each of the electrodes E01-E08) are shown. The stimulation according to each of the trial parameter sets may be scored based on its effect for the patient on the basis of patient responses to the stimulation, as is known in the art. According to some embodiments, the patient responses may include one or more of speech, tremor, rigidity, finger tapping, toe tapping, bradykinesia, hypokinesia, agility posture, gate, postural stability, or the like. In the illustrated example, such effects are referred to as “therapeutic effects,” (TE1-TEn). The accumulated database may also include scores for side effects for each of the trial parameter sets for the patient (shown as SE1 and SE2 in the illustration). Examples of side effects may include paresthesia, tremor, discomfort, and the like. The illustrated embodiment of the accumulated database also includes data for SFMs determined for each of the trial parameter sets used in the various fitting sessions for the various patients. In the illustration, the SFM data is comprised within files of voxelized data for the SFMs. In particular, the illustrated accumulated database comprises raw voxelized SFM data and weighted voxelized SFM data. This will be explained in more detail below. But here it should be appreciated that the accumulated database provides correlations between trial parameter sets that provide good therapeutic outcomes (and/or that cause side effects) and SFMs for those trial parameter sets. Methods of associating SFMs with their therapeutic effects are also discussed in U.S. Pat. No. 11,577,069, the entire contents of which are incorporated herein by reference.

FIG. 9 illustrates an algorithm 900 that can be used to predict effective therapeutic stimulation parameters (or stimulation parameters that may cause side effects) for a subject patient based on an accumulated database, as shown in FIG. 8. At step 902, the algorithm determines stimulation parameter sets that have provided good therapeutic results (or side effects) during the fitting sessions of historic patients contained within the database. For example, the database may be scanned for parameter sets that were shown to be effective at treating symptoms similar to those presented in the subject patient.

At step 904, SFMs of the identified parameter sets are aggregated. The aggregation process involves overlaying the voxelized SFMs, typically within a three dimensional voxel space. Step 906 involves determining where the aggregated SFMs overlap, i.e., determining where some number of the aggregated SFMs occupy the same volume within the voxel space. According to some embodiments, the determination of overlap may be based on a threshold. For example, the overlap region may be considered as a volume occupied by some threshold percentage of the aggregated SFMs. According to some embodiments, overlap determination may be determined using an aggregation algorithm that may be automated.

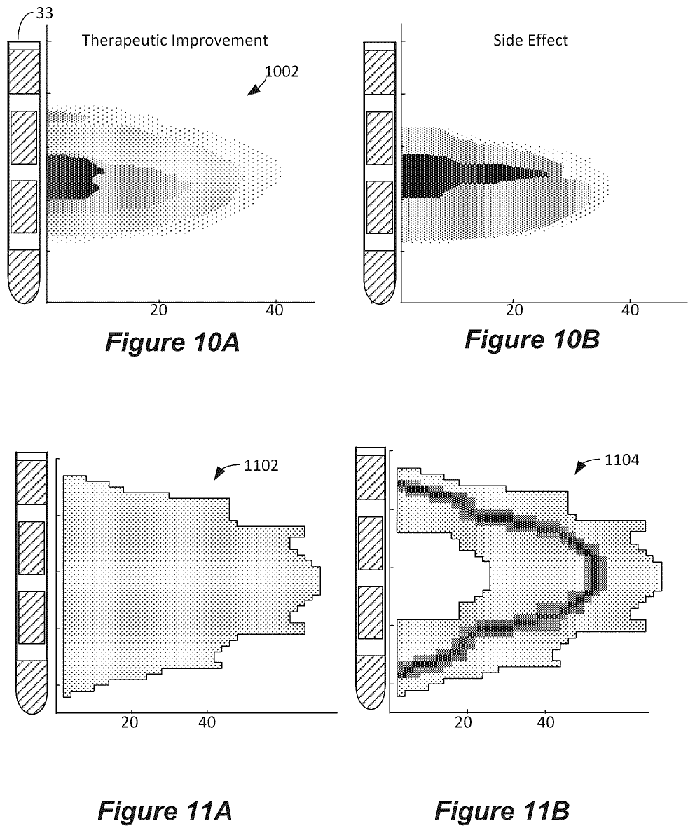

FIGS. 10A and 10B show two dimensional representations of overlap of aggregated SFMs for accumulated stimulation parameters that were therapeutically effective (FIG. 10A) and for stimulation parameters that led to side effects (FIG. 10B) in historic fitting sessions in the accumulated database. It should be noted that FIGS. 10A and 10B are two dimensional representations, but generally, overlap of the three dimensional SFMs comprise a three dimensional voxel space. Each of the representations shown in FIGS. 10A and 10B comprise a representation of a voxel space 1002. Distance from the electrode lead 33 is shown along the x-axis and distance along the lead is shown on the y-axis. According to some embodiments, each voxel in the voxel space may be treated as a histogram, wherein a value is associated with the voxel that indicates the number of SFMs that occupy that voxel. In FIGS. 10A and 10B, darker regions correspond to voxels having higher numbers and lighter regions correspond to voxels having lower numbers. Thus, the areas show regions of a high degree of overlap and lighter areas show regions where less overlap between the SFMs occur.

Visualizations (and/or mathematical analysis, for example, performed using an aggregation algorithm) of the overlap of aggregated SFMs for stimulation parameters associated with therapeutic results and/or side effects derived from the accumulated database can aid the programming of stimulation parameters for a subject patient (FIG. 9, step 908). For example, the clinician may choose to try stimulation parameters for the subject patient that provide stimulation within a region where a high degree of overlap occurs in the aggregated therapeutic SFMs from the accumulated database (i.e., within the dark areas shown in FIG. 10A). In other words, the high overlap region may be considered a target volume for stimulation for the subject patient. Likewise, the clinician may seek to avoid stimulation parameters that stimulate within the high-overlap areas associated with side effects. That region may be considered an avoidance volume for the subject patient. The above incorporated U.S. Pat. No. 8,849,411 discloses methods of using current steering and parameter selection to selectively target stimulation to specific therapy regions. According to some embodiments, the accumulated and overlayed SFMs may be projected upon representations of the subject patient's anatomy using a GUI. Such representations may be derived from imaging data, as is known in the art. Such overlays allow a clinician to correlate the areas of high overlap with specific portions of the patient's anatomy, for example, portions of the patient's STN that might be associated with effective therapy and/or with side effects.

The inventor has recognized that the overlap of SFMs aggregated from the accumulated database is generally biased towards the region closer to the lead. Notice that in both FIGS. 10A and 10B, the highest region of overlap is near to the electrode lead. That is because the stimulation field for each of the stimulation parameters emanate from the electrode lead. Accordingly, an aspect of the instant disclosure provides methods and systems for compensating for the biasing of the aggregated SFMs near to the stimulation lead.

FIG. 11A illustrates a voxelized SFM 1102 calculated for a give stimulation parameter set. Again, it should be appreciated that the SFM is illustrated in two dimensions, but typically the SFM will comprise a three-dimensional volume, which can be voxelized to yield a three dimensional voxel space. Notice that the SFM substantially occupies the region near the electrode lead. When multiple SFMs, each substantially occupying a region near the electrode lead, are overlaid, then the overlap will be greatest near the electrode lead. According to some embodiments, the SFMs contained within the accumulated database can be weighted to emphasize some voxels within the SFM compared to other voxels. For example, the SFMs can be weighted to compensate for the near-electrode lead biasing inherent in the aggregation process. FIG. 11B illustrates an example of a weighted SFM 1104 voxel space. The weighted SFM 1104 is derived by operating on the raw voxelized SFM 1102 using a weighting function that associates weighting values with each of the voxels in the voxel space. Larger weighting values can be used to emphasize given voxels and lower weighting values de-emphasize the voxel. In the weighted SFM 1104, larger weighting values are associated with voxels near the periphery of the SFM. In FIG. 11B, the darker areas of the SFM are more heavily weighted, i.e., those voxels are associated with a larger weighting value. Notice that in the weighted SFM 1104, the region near the electrode lead is de-emphasized, whereas the region near the periphery is emphasized. It should be appreciated that one or more of several features of the SFM may be used for the weighting. As mentioned above, the SFMs may be considered as models representing a probability of a region inside the SFM being activated by the stimulation. In the illustrated example, that probability is highest near the electrode lead and the probability decreases as the distance between the voxels and the lead increases. According to some embodiments, a probability threshold may be used to determine the contour on which the weighting function may be applied. According to some embodiments, the raw voxelized SFM may be weighted by operating on the voxel space using a kernel weighting function, for example, a convolution, a parabolic function, a Gausian function, a quartic function, or the like. According to some embodiments, the weighted SFMs for each of the stimulation parameter sets determined over a plurality of historic fitting sessions may be stored in the accumulated database 720, as illustrated in FIG. 8.

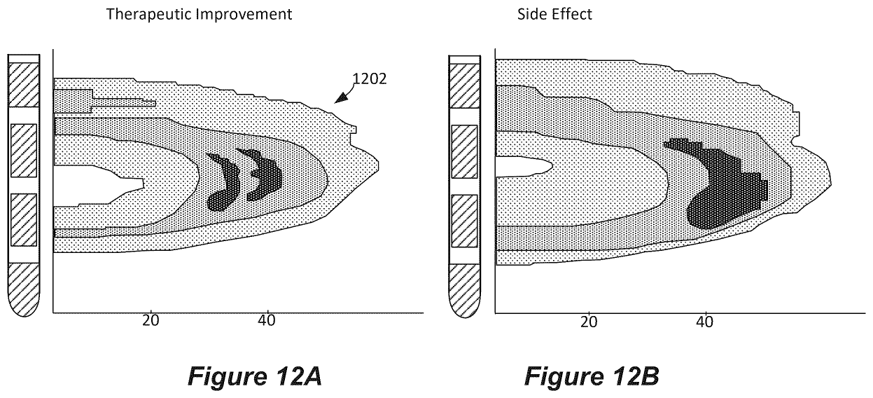

FIGS. 12A and 12B illustrate aggregated weighted SFMs from an accumulated database corresponding to stimulation parameters showing therapeutic effectiveness (FIG. 12A) and linked to side effects (FIG. 12B). As with the aggregated SFMs illustrated in FIGS. 10A and 10B discussed above, the aggregated weighted SFMs shown in FIGS. 12A and 12B each comprise a voxel space 1202. Each of the voxels in the voxel space of the aggregation is associated with a value that is indicative of 1) the number of aggregated weighted SFMs that occupy that voxel, and 2) the weight associated with that voxel in each of the occupying SFMs. In FIGS. 12A and 12B, the darker portions correspond to higher degrees of effective (i.e., “weighted”) overlap of the aggregated SFMs. Notice that the voxels near the electrode lead are associated with lower effective values, even though the raw SFMs have a high degree of overlap in that region. The lower effective values near the lead result because the weighting values associated with that region in the component SFMs are smaller. Thus, those regions are de-emphasized when the weighted SFMs are aggregated. A clinician seeking to program stimulation parameters to maximize therapeutic effectiveness may concentrate on stimulation parameter sets that focus stimulation in the areas indicated by the darkest regions of FIG. 12A. Likewise, to minimize side effects, the clinician may focus on stimulation parameters that avoid stimulating the darkest regions of FIG. 12B. As mentioned above, representations such as shown in FIGS. 12A and 12B may be overlayed with imaging data from the subject patient (or representations derived therefrom) to correlate the high overlap regions with aspects of the patient's anatomy.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the method comprising:
receiving accumulated data from a database, wherein the accumulated data comprises:
previous trial stimulation parameter sets provided to previous patients or the subject patient,
stimulation field models (SFMs) for each of the trial stimulation parameter sets, and
scores indicative of the therapeutic effectiveness of each of the trial stimulation parameter sets for the previous patients,
aggregating the SFMs of each of the trial stimulation parameter sets having a score exceeding a threshold score or within a defined threshold range,
determining an overlap of the aggregated SFMs, and
using the overlap to determine electrical stimulation parameters for the subject patient.

2. The method of claim 1, wherein aggregating the SFMs comprise voxelizing the SFMs having a score exceeding the threshold score and overlaying the voxelized SFMs in a common voxel space.

3. The method of claim 2, wherein determining an overlap of the aggregated SFMs comprises determining overlap values for each of the voxels of the common voxel space, wherein the overlap values for each of the voxels are indicative of the number of aggregated SFMs occupying that voxel of the common voxel space.

4. The method of claim 2, further comprising using a kernel weighting function to associate a weighting value with each of the voxels of the voxelized SFMs.

5. The method of claim 4, wherein each of the voxelized SFMs comprise voxels associated peripheral regions of the SFM and voxels associated with regions near an electrode lead from which the SFM emanates.

6. The method of claim 5, wherein the weighting values associated with voxels associated peripheral regions of the SFM are greater than the weighting values associated with voxels associated with regions near an electrode lead from which the SFM emanates.

7. The method of claim 4, wherein determining an overlap of the aggregated SFMs comprises determining overlap values for each of the voxels of the common voxel space, wherein the overlap values for each of the voxels are indicative of the number of aggregated SFMs occupying that voxel of the common voxel space and of the weighting values associated with corresponding voxels of the aggregated SFMs.

8. The method of claim 1, further comprising displaying an indication of the overlap of the aggregated SFMs on a graphical user interface (GUI).

9. The method of claim 1, wherein using the overlap to determine electrical stimulation parameters for the subject patient comprises determining stimulation parameters that provide stimulation to a region where the aggregated SFMs overlap.

10. The method of claim 1, wherein using the overlap to determine electrical stimulation parameters for the subject patient comprises determining stimulation parameters that provide stimulation to a region where the overlap of the aggregated SFMs exceed a threshold.

11. A system for programming electrical stimulation parameters for providing deep brain stimulation (DBS) to a subject patient, wherein the subject patient is implanted with an implantable medical device comprising an implantable pulse generator (IPG) connected to one or more electrode leads implanted in the subject patient's brain, wherein each electrode lead comprises a plurality of electrodes, the system comprising:
an external computing device comprising control circuitry configured to perform a method, the method comprising:
receiving accumulated data from a database, wherein the accumulated data comprises:
previous trial stimulation parameter sets provided to previous patients or the subject patient,
stimulation field models (SFMs) for each of the trial stimulation parameter sets, and
scores indicative of the therapeutic effectiveness of each of the trial stimulation parameter sets for the previous patients,
aggregating the SFMs of each of the trial stimulation parameter sets having a score exceeding a threshold score or within a defined threshold range,
determining an overlap of the aggregated SFMs, and
using the overlap to determine electrical stimulation parameters for the subject patient.

12. The system of claim 11, wherein aggregating the SFMs comprise voxelizing the SFMs having a score exceeding the threshold score and overlaying the voxelized SFMs in a common voxel space.

13. The system of claim 12, wherein determining an overlap of the aggregated SFMs comprises determining overlap values for each of the voxels of the common voxel space, wherein the overlap values for each of the voxels are indicative of the number of aggregated SFMs occupying that voxel of the common voxel space.

14. The system of claim 12, wherein the method further comprises using a kernel weighting function to associate a weighting value with each of the voxels of the voxelized SFMs.

15. The system of claim 14, wherein each of the voxelized SFMs comprise voxels associated peripheral regions of the SFM and voxels associated with regions near an electrode lead from which the SFM emanates.

16. The system of claim 15, wherein the weighting values associated with voxels associated peripheral regions of the SFM are greater than the weighting values associated with voxels associated with regions near an electrode lead from which the SFM emanates.

17. The system of claim 14, wherein determining an overlap of the aggregated SFMs comprises determining overlap values for each of the voxels of the common voxel space, wherein the overlap values for each of the voxels are indicative of the number of aggregated SFMs occupying that voxel of the common voxel space and of the weighting values associated with corresponding voxels of the aggregated SFMs.

18. The system of claim 11, wherein the method further comprises displaying an indication of the overlap of the aggregated SFMs on a graphical user interface (GUI).

19. The system of claim 11, wherein using the overlap to determine electrical stimulation parameters for the subject patient comprises determining stimulation parameters that provide stimulation to a region where the aggregated SFMs overlap.

20. The system of claim 11, wherein using the overlap to determine electrical stimulation parameters for the subject patient comprises determining stimulation parameters that provide stimulation to a region where the overlap of the aggregated SFMs exceed a threshold.
